(19) United States
(12) Patent Application Publication
(10) Pub. No.: US 2003/0073911 A1
(43) Pub. Date: Apr. 17, 2003

(54) Pace control device

(76) Inventors: Manabu Yoshimura, Kyoto-shi (JP); Shui-Jung Chen, Taipei Hsien (TW)

Correspondence Address: RABIN & BERDO, P.C., Suite 500, 1101 14th Street, N.W., Washington, DC, 20005, US

(21) Appl. No.: 09/977,987

(22) Filed: Oct. 17, 2001

(51) Int. Cl. A61B 5/02; A61B 5/04

(52) U.S. Cl. 600/500; 600/520

(57) ABSTRACT

The present invention provides a pace control device comprising a chest belt sensor device and a data receiver. When the chest belt sensor device is attached to the chest of the user, the acceleration sensor measures the workout paces, and the heart rate sensor circuit measures the heart rate. These measures are sent to the microprocessor to be encoded by an ID coded protocol and then transmitted by the wireless transmitter circuit to the data receiver. The data receiver comprises a wireless receiver circuit, a monitor, and a microprocessor with the same ID coded protocol as in the chest belt sensor device. The data receiver obtains the data on the heart rate and the workout paces and displays the information on the monitor so that the user can adjust their working at an optimum level.

### BACKGROUND OF THE INVENTION

I. Field of the Invention

This invention relates generally to a pace control device and, more specifically, to a pace control device which is to be attached to the chest when the user is doing exercise and can offer immediate measurements on the workout paces and the heart rate information to the user. The heart rate increases when the workout paces go up, and the heart rate decreases when the workout paces go down. The present invention detects and measures the workout paces and the heart rate and gives immediate feedback so that the user can track the aerobic exercise level and make necessary adjustment to achieve the desired heart rate target zone.

II. Description of the Prior Art

Heretofore, it is known to construct a heart rate monitor having a telemetry transmitter and a wireless receiver watch, as shown in FIG. 4. A heart rate monitor of such construction is to be attached to the chest of the user, with an EKG device measuring the heart rate and the telemetry transmitter transferring the data to the wireless receiver watch. The user can read the heart rate information from the monitor of the wireless receiver watch (U.S. Pat. No. 5,486,818).

Nonetheless, a heart rate monitor of this kind typically provides only heart rate information and does not give the feedback on the workout paces. Without the information on workout paces, the user would have difficulty tracking the aerobic exercise level and thus may not be able to keep the working in a safe zone and at the same time achieve the target zone.

It is also known to construct a heart rate monitor in terms of a pedometer, which measures workout paces and transmits these data to a data receiver watch through a wireless transmitting device. The user can read the data right from the monitor on the data receiver watch (refer to U.S. Pat. No. 6,160,480). Nonetheless, a heart rate monitor of this kind typically provides only feedback on the workout paces and does not give the heart rate information. In order to provide information on both workout paces and heart rate, two separate devices have to be combined so that the user would not have to carry both devices to obtain the desired information.

### SUMMARY OF THE INVENTION

It is therefore a primary object of the invention to provide a pace control device measuring both the heart rate and the workout paces and transferring these data to a data receiver in a wireless manner. These data are to be displayed on the screen of the data receiver, based on which the user can adjust the working level to achieve competitive performance.

The present invention improves on the heretofore known heart rate monitor by providing a chest belt sensor device comprising an acceleration sensor, a heart rate sensor circuit (with the technique of EKG), a microprocessor and a wireless transmitter circuit. The chest belt sensor device is to be attached to the chest when the user is doing exercise. The acceleration sensor detects and measures the workout paces, and the heart rate sensor circuit measures the heart rate, with both signals sent to the microprocessor to be encoded by the ID coded protocol. The encoded data are then transmitted by the wireless transmitter circuit to the data receiver. The data receiver comprises a wireless receiver circuit, a monitor, and a microprocessor with the ID coded protocol same as the one in the microprocessor of the chest belt sensor device. Once receiving data from the microprocessor of the chest belt sensor device, the data receiver displays the information on heart rate and workout paces on the monitor.

With the acceleration sensor measuring the workout paces and the heart rate sensor circuit measuring the heart rate, the information can be processed and passed wirelessly to the data receiver, where the user can read the information from the monitor, according to which the user can make adjustment on the working level to achieve the target zone.

### BRIEF DESCRIPTION OF THE DRAWINGS

An embodiment of the invention will now be described by way of example only, with reference to the accompanying drawing, in which:

FIG. 1 is a schematic drawing of an embodiment of the present invention;

FIG. 2 is a schematic drawing of an acceleration sensor according to the present invention;

FIG. 3 is a schematic drawing of ID coded protocol of the present invention;

FIG. 4 is a circuit diagram showing the complete circuit according to the present invention.

FIG. 5 is a circuit block diagram showing the heart rate sensor circuit according to the present invention.

### DESCRIPTION OF THE PREFERRED EMBODIMENT

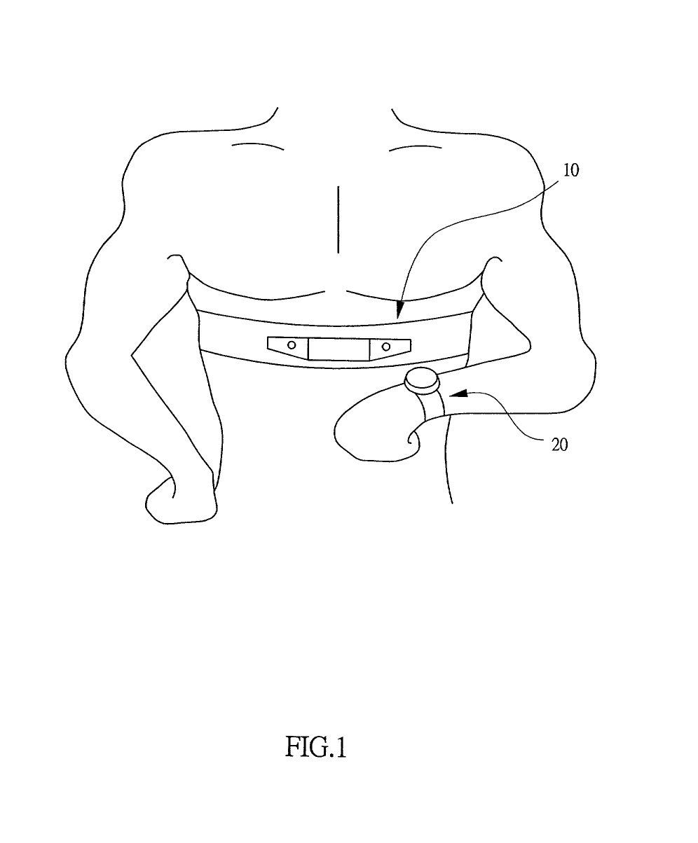

Refer from FIG. 1 to FIG. 4, the present invention includes a chest belt sensor device and a data receiver 20. The chest belt sensor device 10 consists of a chest belt 11 and a sensor shell 12 in the middle of the chest belt 11. Inside the sensor shell 12 are an acceleration sensor 13, a heart rate sensor circuit 14, a microprocessor 15 and a wireless transmitter circuit 16.

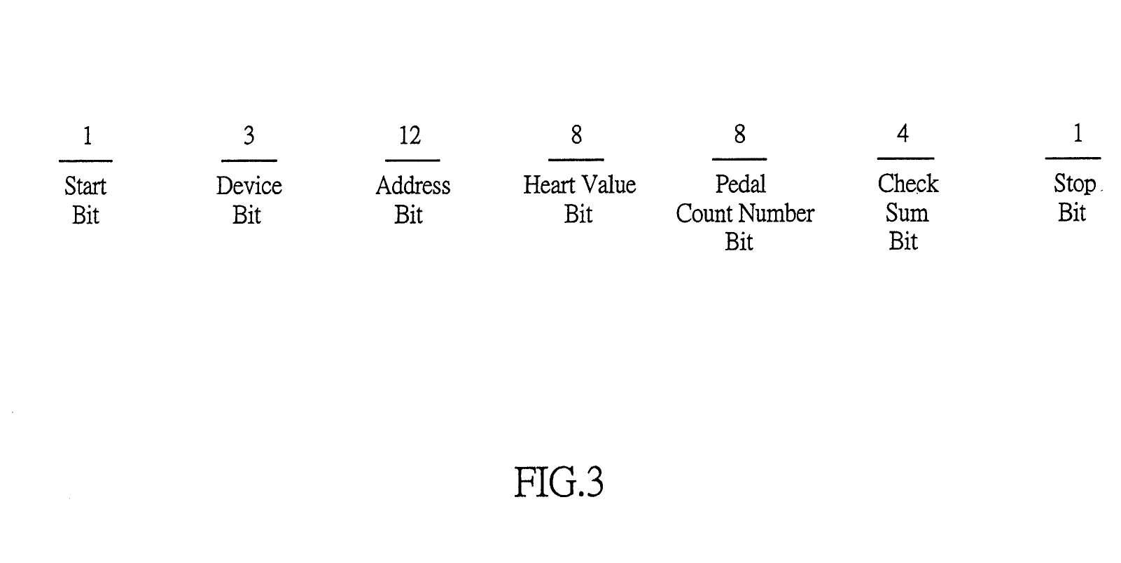

When chest belt sensor device 10 is attached to the chest of the user during exercise, the acceleration sensor 13 detects and measures the workout paces, and the heart rate sensor circuit 14 measures the heart rate. Both of the measures are passed to the microprocessor 15 to be encoded by the ID coded protocol (as shown in FIG. 3) and transmitted by the wireless transmitter circuit 16 to the data receiver 20. The data receiver 20 comprises a wireless receiver circuit 21, a monitor 22 and a microprocessor 23 with the same ID coded protocol as in the microprocessor 15 of the chest belt sensor device 10. Once receiving the data, the data receiver 20 displays the information of paces and heart rate on the monitor 22 for the reference of users in order to adjust their working at an optimum level.

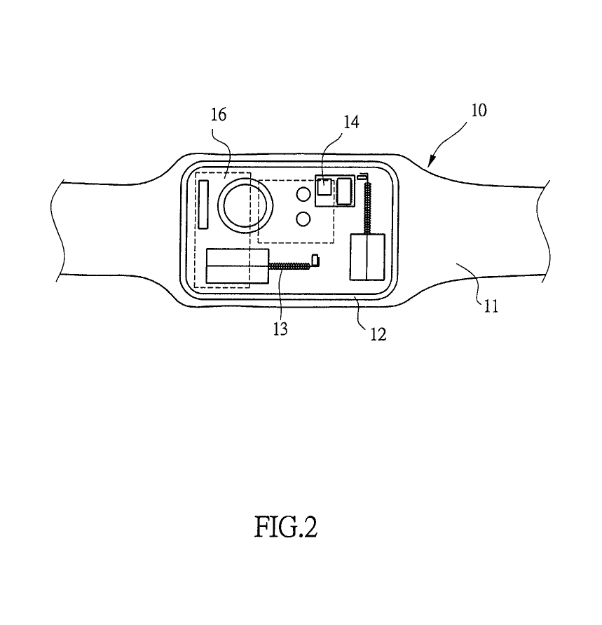

The acceleration sensor 13 is designed as having two dimensions (X- and Y-axis) or three dimensions (X-, Y- and Z-axis). As shown in FIG. 2, the acceleration sensor 13 is installed inside the sensor shell 12 of the chest belt sensor device 10 and can accurately measure the workout paces during exercise. The data of workout paces are encoded and transmitted wirelessly to the data receiver 20, where the user can read this information from the monitor 22 of the data receiver 20.

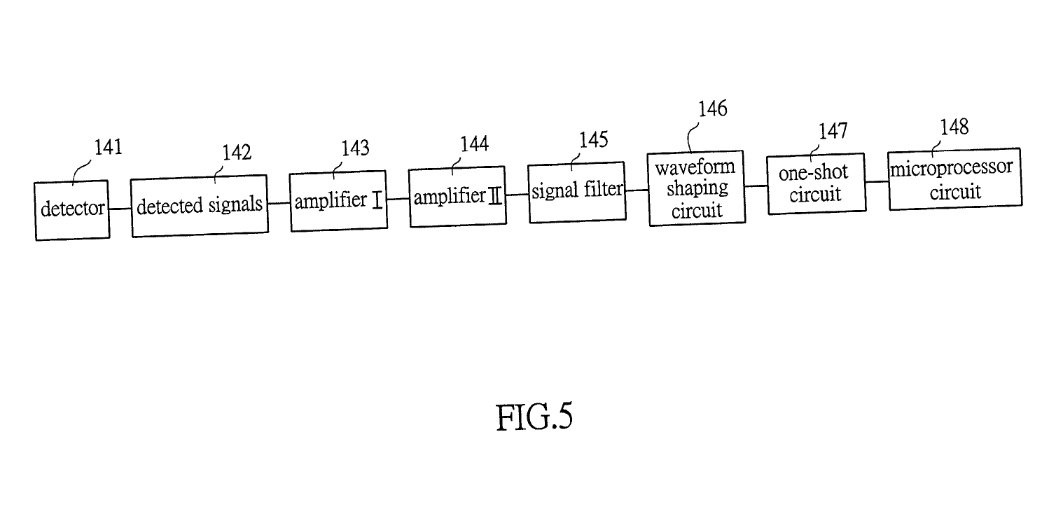

Refer to FIG. 5, the heart rate sensor circuit 14 adopts the technique of EKG to measure heart rate, as shown in FIG. 5. The detected signals 142 obtained by the detector 141 are amplified by the signal amplifiers 143, 144, processed by the signal filter 145 and the waveform shaping circuit 146, and transmitted out through the one-shot circuit 147 and the microprocessor 148.

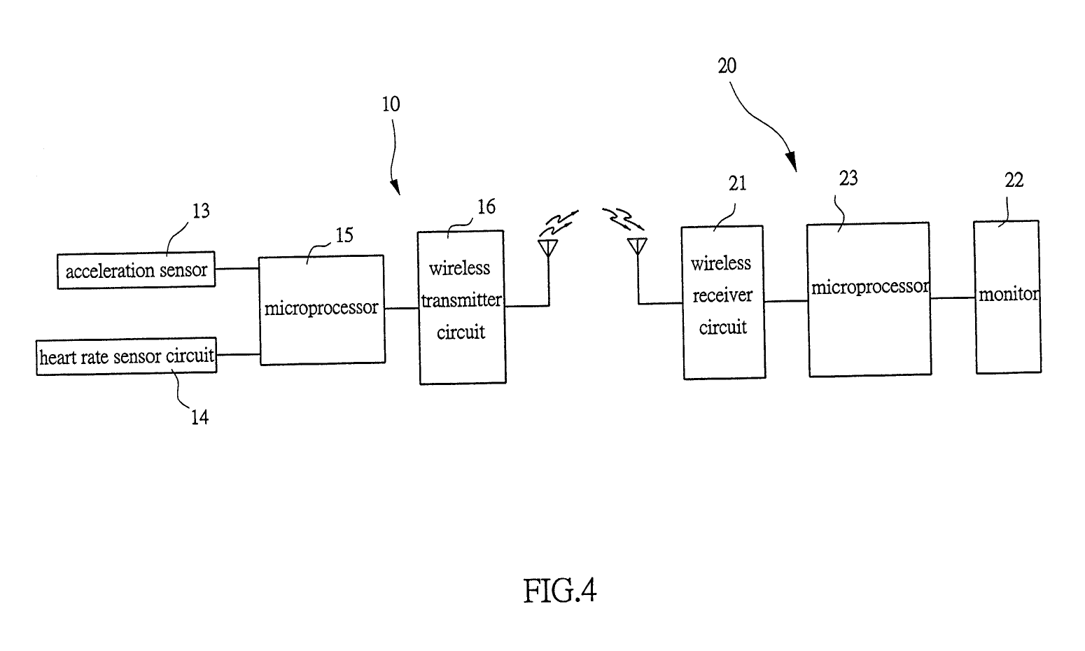

Also refer to FIG. 4, the chest belt sensor device 10 can recode two different signals with the same ID coded protocol and thus transmit the data in a wireless manner. The data receiver 20 can be in the form of a watch, a conventional pedometer or a pager.

The invention features on that the acceleration sensor 13 measures workout paces, and the heart rate sensor circuit 14 measures heart rate simultaneously. After being encoded by the microprocessor 15, these measures are transmitted to the data receiver 20 and shown on the monitor 22. Based on the information on both heart rate and workout paces, the user is able to make necessary adjustment to keep working at an optimum level.

While only one embodiment of the present invention has been shown and described, it will be understood that various modifications and changes could be made thereunto without departing from the spirit and scope of the invention disclosed.

What is claimed is:

1. A pace control device comprising a chest belt sensor device and a data receiver, said chest belt sensor device having an acceleration sensor, a heart rate sensor circuit, a microprocessor and a wireless transmitter circuit;
said data receiver having a wireless receiver circuit, a monitor and a microprocessor;
wherein said chest belt sensor device is attached to the chest of users, said acceleration sensor detects and measures workout paces of users while said heart rate sensor circuit measures heart rate of users; and both of signals of workout paces and heart rate are passed to said microprocessor of said chest belt sensor device, then being transmitted to the data receiver by said wireless transmitter circuit; once receiving the signal, said data receiver displays the information of workout paces and heart rate on said monitor.

2. A pace control device as claimed in claim 1, wherein said acceleration sensor is designed as having two dimensions (X- and Y-axis)or three dimensions (X-, Y- and Z-axis).

3. A pace control device as claimed in claim 1, wherein said chest belt sensor device and said data receiver having microprocessors with the same ID coded protocol.

4. A pace control device as claimed in claim 1, wherein said data receiver can be in the form of a watch, a pedometer or a pager.
